(19) United States
(12) Patent Application Publication
(10) Pub. No.: US 2006/0052865 A1
(43) Pub. Date: Mar. 9, 2006

(54) Stents with metallic covers and methods of making same

(76) Inventor: Christopher E. Banas, San Antonio, TX (US)

Correspondence Address: ROSENBAUM & ASSOCIATES, P.C.;Suite #380, 650 Dundee Road, Northbrook, IL, 60062, US

(21) Appl. No.: 10/936,883

(22) Filed: Sep. 9, 2004

(51) Int. Cl. A61F 2/06

(52) U.S. Cl. 623/1.44

(57) ABSTRACT

All metal stent grafts and covered stents having either a single structural supporting stent member with concentrically positioned graft members on the luminal and abluminal surfaces of the stent member or a single graft member with concentrically positioned structural supporting stent members on the luminal and abluminal surfaces of the graft member are provided.

### CROSS-REFERENCE TO RELATED INVENTIONS

The present application is related to commonly assigned, co-pending U.S. application Ser. No. 10/135,136 filed Apr. 29, 2002, which claims priority from U.S. Ser. No. 60/310,617, filed Aug. 7, 2001, U.S. Ser. No. 09/7455,304, filed Dec. 22, 2000 which is a divisional of Ser. No. 09/443,929, filed Nov. 19, 1999, now U.S. Pat. No. 6,379,383, and U.S. Ser. Nos. 10/289,974 and 10/289,843 09/532,164 both filed Nov. 6, 2002 which are continuation applications of U.S. Ser. No. 09/532,164, filed Mar. 20, 2000, now U.S. Pat. No. 6,537,310, each of which are hereby incorporated by reference.

### BACKGROUND OF THE INVENTION

The present invention pertains generally to the field of medical devices intended to maintain patency of anatomical passageways, such as those found in the cardiovascular, lymphatic, endocrine, renal, gastrointestinal and/or reproductive systems of mammals. More particularly, the present invention relates to stents, stent-grafts and covered stents that are designed for endoluminal delivery using a delivery catheter and minimally invasive surgical techniques. The present invention generally comprises stent-graft or covered stent type devices that are fabricated entirely of biocompatible metals or of biocompatible materials that exhibit biological response and material characteristics substantially the same as biocompatible metals, such as for example composite materials. For purposes of the present application, the terms “stent-graft” and “covered stent” are used interchangeably.

Conventional endoluminal stents and stent-grafts are frequently used in conjunction with a procedure which dilitates an occluded, obstructed or diseased anatomical passageway to provide structural support and maintain the patency of the anatomical passageway. An example of this is the post-angioplasty use of intravascular stents to provide a structural support for a blood vessel and reduce the incidence of restenosis. A principal, but non-limiting, example of the present invention are endovascular stents which are introduced to a site of disease or trauma within the body's vasculature from an introductory location remote from the disease or trauma site using an introductory catheter, passed through the vasculature communicating between the remote introductory location and the disease or trauma site, and released from the introductory catheter at the disease or trauma site to maintain patency of the blood vessel at the site of disease or trauma. Stent-grafts are delivered and deployed under similar circumstances and are utilized to maintain patency of an anatomic passageway, for example, by reducing restenosis following angioplasty, or when used to exclude an aneurysm, such as in aortic aneurysm exclusion applications.

While the use of endoluminal stents has successfully decreased the rate of restenosis in angioplasty patients, it has been found that a significant restenosis rate continues to exist even with the use of endoluminal stents. It is generally believed that the post-stenting restenosis rate is due, in major part, to the non-regrowth of a healthy endothelial layer over the stent and the incidence of smooth muscle cell-related neointimal growth on the luminal surfaces of the stent. Injury to the endothelium, the natural nonthrombogenic lining of the arterial lumen, is a significant factor contributing to restenosis at the situs of a stent. Endothelial loss exposes thrombogenic arterial wall proteins, which, along with the generally thrombogenic nature of many prosthetic materials, such as stainless steel, titanium, tantalum, Nitinol, etc. customarily used in manufacturing stents, initiates platelet deposition and activation of the coagulation cascade, which results in thrombus formation, ranging from partial covering of the luminal surface of the stent to an occlusive thrombus. Additionally, endothelial loss at the site of the stent has been implicated in the development of neointimal hyperplasia at the stent situs. Accordingly, rapid re-endothelialization of the arterial wall with concomitant endothelialization of the body fluid or blood contacting surfaces of the implanted device is considered critical for maintaining vasculature patency and preventing low-flow thrombosis.

At present, most endoluminal stents are manufactured of either stainless steel or nickel-titanium alloy, both of which are known to be thrombogenic. In order to reduce the thrombogenicity of the stainless steel and to maintain sufficient dimensional profiles for catheter delivery, most stents minimize the metal surface area that contacts blood, in order to minimize thrombus formation after implantation. Thus, in order to reduce the thrombogenic response to stent implantation, as well as reduce the formation of neointimal hyperplasia, it would be advantageous to increase the rate at which endothelial cells form endothelium proximal and distal to the stent situs, migrate onto and provide endothelial coverage of the luminal surface of the stent which is in contact with blood flow through the vasculature.

Stent-grafts are essentially endoluminal stents with a discrete covering on either or both of the luminal and abluminal surfaces of the stent that occludes the open spaces, or interstices, between adjacent structural members of the endoluminal stent. It is known in the art to fabricate stent-grafts by covering the stent with endogenous vein or a synthetic material, such as woven polyester known as DACRON, or with expanded polytetrafluoroethylene. Additionally, it is known in the art to cover the stent with a biological material, such as a xenograft or collagen. A primary purpose for covering stents is to reduce the thrombogenic effect of the stent material and reduce particulate extrusion through interstices of the stent and into the bloodstream. Conventional graft materials have not proven to be a complete solution to enhancing the healing response of conventional stents.

Heretofore, the art has not provided a stent-graft device in which a structural component, such as a stent, and a graft component are each fabricated of biocompatible metals or of biocompatible materials which exhibit in vivo biological and mechanical responses substantially the same as biocompatible metals (hereinafter synonymously referred to as “pseudometals” or “pseudometallic materials”).

### SUMMARY OF THE INVENTION

It is, therefore, a principal objective of the present invention to provide a stent-graft type device fabricated entirely of biocompatible metals and/or pseudometallic materials. That is, the inventive stent-graft type device consists generally of a structural component, e.g., a stent, a plurality of stents, or a plurality of support structures, and a covering component, e.g., a graft, each of which is formed of a metal or pseudometal. The structural component will be referred to hereinafter for ease of reference, will be termed a “stent”, while the covering component will similarly be termed a “graft” for ease of reference. Those of ordinary skill in the art will appreciate, however, that the terms “structural component” and “covering component” have broader meaning and encompass a wide variety of structures other than stents or grafts.

The stent may consist of any type of structural member and is preferably generally tubular in configuration, and has an inner or luminal wall and an outer or abluminal wall and a central lumen passing along the longitudinal axis of the stent. The stent may be comprised of a wide variety of geometric configurations and constructions, as are known in the art. For example, the stent may assume a balloon expandable slotted configuration of U.S. Pat. Nos. 4,733,665, 4,739,762, 4,776,337 or 5,102,417 or the stent may be configured as a plurality of self-expanding interwoven wire members or it may assume any of the wall geometries disclosed in Serruys, P. W., Kutryk, M. J. B., Handbook of Coronary Stents, 3^{rd} Ed. (2000). Each of the stent designs, stent materials, stent material characteristics, e.g., balloon expandable, self-expanding by spring tension of the material, self-expanding by shape memory properties of the stent material, or self-expanding by superelastic properties of the stent material are well known to one of ordinary skill in the art and may be used with the stent-graft of the present invention.

The covering component, or the graft of the present invention may be employed on either or both of the luminal wall and/or the abluminal wall of the stent, and may cover all or a portion of either or both of the luminal and/or abluminal walls of the stent. The graft may be formed as a planar film of material that is applied to the stent by wrapping about stent or formed into a tubular structure and coupled to the stent. Alternatively the graft may be formed as an integral tubular member that is coupled to the stent. The graft may also be fashioned as at least two graft members, with a first graft member covering a luminal wall surface of the stent and a second graft member covering an abluminal wall surface of the stent. Alternatively, the graft may be formed as a single member that covers both a luminal wall surface and an abluminal wall surface of the stent and is everted over either or both ends of the stent. Further, the graft member may be joined to the stent, or where a graft covers both the luminal and abluminal wall surfaces of the stent, the graft may be joined to an opposing graft surface through interstices in the stent. The juncture between the graft and the stent or the graft to the graft through the stent may be accomplished by chemical, mechanical or thermal means such as by welding, adhesion using biocompatible adhesives, interference fits, interlocking or interfacing couplings, such as an interfacing detent and trough combination, or such other methods of joining or coupling metallic and pseudometallic materials to itself or one another as are known in the art. Where an interfacing detent-trough combination is employed as a coupling, it may be a direct interface or it may server to lock the graph material into a fixed position relative to the structural support members. Further, in order to minimize percent strain resulting from the coupling of a trough and detent, it is desirable that the detent and trough have radiused surfaces.

The structural support component and the covering component are preferably fabricated entirely of self-supporting films made of biocompatible metals or biocompatible pseudometals. The metal films may either be single layer metal films or plural layer films. The terms “metal film,” “thin metallic film” and “metal thin film” are used in this application synonymously to refer to single or plural layer films fabricated of biocompatible metals or biocompatible pseudometals having thicknesses greater than 0 μm and less than about 125 μm. When used as the structural support component, the thin metallic film preferably has a thickness greater than about 25 μm and when used as the covering component, the thin metallic film preferably has a thickness between 0.1 μm and 25 μm and most preferably between 0.1 μm and 10 μm.

There are generally two embodiments of the inventive stent-graft. A first embodiment consists of a stent covered by graft material on each of the luminal and abluminal wall surfaces of the stent. A second embodiment consists of a first and second stent members concentrically positioned coaxial with one another, with at least one graft member concentrically positioned intermediate the first and second stent members. In this second embodiment, the at least one graft member may further encapsulate one or both of the first and second stent members.

In accordance with an inventive method for making the inventive stent-graft, at least one discrete graft member may be conjoined with a plurality of structural members, such as a stent, by joining or coupling the graft member to regions of the structural members. The joined regions may be at a proximal and/or a distal end of the device, or may be at intermediate regions along the longitudinal and circumferential axes of the device. Alternatively, where the graft member or members cover both the luminal and abluminal wall surfaces of the structural members, the graft member or members may be mechanically joined to one another through interstices formed between adjacent pairs of structural members. An alternative method for making the inventive stent-graft includes employing vacuum deposition methodologies, such as those employed in the microelectronics fabrication arts. For example sputtering, physical vapor deposition, ion beam-assisted evaporative deposition or the like, may be used to create either or both of the graft and the stent components of the inventive stent-graft device. In ion beam-assisted evaporative deposition it is preferable to employ dual and simultaneous thermal electron beam evaporation with simultaneous ion bombardment of the material being deposited using an inert gas, such as argon, xenon, nitrogen or neon. Bombardment with inert gas ions during deposition serves to reduce void content by increasing the atomic packing density in the deposited material. The reduced void content in the deposited material allows the mechanical properties of that deposited material to be similar to the bulk material properties. Deposition rates up to 20 nm/sec are achievable using ion beam-assisted evaporative deposition techniques.

When sputtering techniques are employed, a 200-micron thick stainless steel film may be deposited within about four hours of deposition time. Thinner films may be achieved by using shorter deposition times. With the sputtering technique, it is preferable to employ a cylindrical sputtering target, a single circumferential source that concentrically surrounds the substrate that is held in a coaxial position within the source.

During deposition, various deposition process parameters, including, without limitation, target composition, target temperature, chamber pressure, deposition pressure, deposition rate, target configuration, target-to-source distance, bias or partial pressure of the process gases are controlled to optimize deposition of the desired species onto the substrate. As is known in the microelectronic fabrication, nano-fabrication and vacuum coating arts, both the reactive and non-reactive gases are controlled and the inert or non-reactive gaseous species introduced into the deposition chamber is typically argon. The substrate may be either stationary or moveable; either rotated about its longitudinal axis, moved in an X-Y plane, planatarily or rotationally moved within the deposition chamber to facilitate deposition or patterning of the deposited material onto the substrate. The deposited material may be deposited either as a uniform solid film onto the substrate, or patterned by (a) imparting either a positive or negative pattern onto the substrate, such as by etching or photolithography techniques applied to the substrate surface to create a positive or negative image of the desired pattern or (b) using a mask or set of masks which are either stationary or moveable relative to the substrate to define the pattern applied to the substrate. Patterning may be employed to achieve complex finished geometries of the resultant structural supports, web-regions or graft, both in the context of spatial orientation of patterns of regions of relative thickness and thinness, such as by varying the thickness of the film over its length to impart different mechanical characteristics under different delivery, deployment or in vivo environmental conditions.

The device may be removed from the substrate after device formation by any of a variety of methods. For example, the substrate may be removed by chemical means, such as etching or dissolution, by ablation, by machining or by ultrasonic energy. Alternatively, a sacrificial layer of a material, such as carbon, aluminum or organic based materials, such as photoresists, may be deposited intermediate the substrate and the stent and the sacrificial layer removed by melting, chemical means, ablation, machining or other suitable means to free the stent from the substrate.

Optionally, the resulting device may then be subjected to post-deposition processing to modify the crystalline structure, such as by annealing, or to modify the surface topography, such as by etching to expose a heterogeneous surface of the device.

Alternate deposition processes which may be employed to form the stent in accordance with the present invention are cathodic arc, laser ablation, and direct ion beam deposition. As known in the metal fabrication arts, the crystalline structure of the deposited film affects the mechanical properties of the deposited film. These mechanical properties of the deposited film may be modified by post-process treatment, such as by, for example, annealing.

Materials to make the inventive graft, stent-graft and web-stent are chosen for their biocompatibility, mechanical properties, i.e., tensile strength, yield strength, and their ease of deposition include, without limitation, the following: elemental titanium, vanadium, aluminum, nickel, tantalum, zirconium, chromium, silver, gold, silicon, magnesium, niobium, scandium, platinum, cobalt, palladium, manganese, molybdenum and alloys thereof, such as zirconium-titanium-tantalum alloys, nitinol, and stainless steel.

The inventive stent-graft type device of the present invention is formed entirely of metal or pseudometallic material that exhibits improved endothelialization and healing response as compared to that associated with using conventional synthetic polymeric graft materials.

### BRIEF DESCRIPTION OF THE DRAWINGS

FIG. 1 is a perspective view of a first embodiment of the stent-graft of the present invention.

FIG. 2 is a cross-sectional view taken along line 2-2 of FIG. 1.

FIG. 3 is a cross-sectional view taken along line 3-3 of FIG. 1.

FIG. 4 is a cross-sectional view of a second embodiment of the stent-graft of the present invention.

FIG. 5 is a cross-sectional view of a third embodiment of the stent-graft of the present invention.

FIG. 6 is a perspective view of a fourth embodiment of the sent-graft of the present invention.

FIG. 7 is a cross-sectional view taken along line 7-7 of FIG. 6.

FIG. 8 is a cross-sectional view taken along line 8-8 of FIG. 6.

FIG. 9 is a cross-sectional view of a fifth embodiment of the stent-graft of the present invention.

FIG. 10 is a cross-sectional view of a sixth embodiment of the stent-graft of the present invention.

FIG. 11 is a flow diagram illustrating fabrication methodologies for making the inventive stent-graft of the present invention.

### DETAILED DESCRIPTION OF THE PREFERRED EMBODIMENTS

In accordance with the present invention there are generally two preferred embodiments of the inventive covered stent assembly. A first embodiment generally comprises a structural support member, such as a stent, that has first and second opposing wall surfaces thereof that are bounded by metallic or pseudometallic covers. The covers are positioned adjacent the first and second opposing wall surfaces and are either coupled to the structural support member or are coupled to one another through interstitial openings passing through the structural support member. The second embodiment generally comprises at least one metallic or pseudometallic cover that is positioned intermediate two adjacent structural support members, e.g., stents. For purposes of this application, the terms “pseudometal” and “pseudometallic” are intended to mean a biocompatible material which exhibits biological response and material characteristics substantially the same as biocompatible metals. Examples of pseudometallic materials include, for example, composite materials, ceramics, quartz, and borosilicate. Composite materials are composed of a matrix material reinforced with any of a variety of fibers made from ceramics, metals, or polymers. The reinforcing fibers are the primary load carriers of the material, with the matrix component transferring the load from fiber to fiber. Reinforcement of the matrix material may be achieved in a variety of ways. Fibers may be either continuous or discontinuous. Reinforcement may also be in the form of particles. Examples of composite materials include those made of carbon fibers, boron fibers, boron carbide fibers, carbon and graphite fibers, carbon nanotubes, silicon carbide fibers, steel fibers, tungsten fibers, graphite/copper fibers, titanium and silicon carbide/titanium fibers.

The structural support member and the covering member are preferably fabricated entirely of self-supporting films made of biocompatible metals or biocompatible pseudometals. The metal films may either be single layer metal films or plural layer films. The terms “metal film,” “thin metallic film” and “metal thin film” are used in this application synonymously to refer to single or plural layer films fabricated of biocompatible metals or biocompatible pseudometals having thicknesses greater than 0 μm and less than about 125 μm.

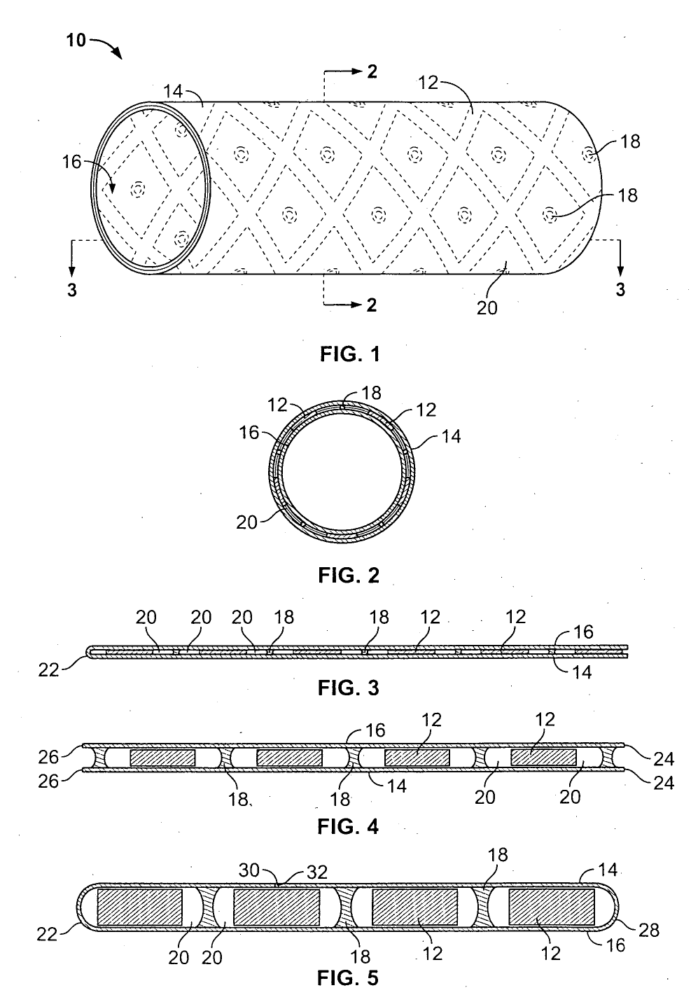

Turning now to the accompanying figures, FIGS. 1-5 illustrate the first embodiment of the present invention and variations thereupon. With particular reference to FIGS. 1 and 2, the inventive stent-graft 10 consists generally of at least one structural support member 12 having a first wall surface 12a and a second wall surface 12b opposing one another, a first cover member 14 and a second cover member 16. The first cover member 14 is positioned adjacent the first wall surface 12a while the second cover member 16 is positioned adjacent the second wall surface 12b of the structural support member 12. The first cover member 14 and the second cover 16 may be coupled either to the structural support member 12 or to one another through interstitial openings 20 passing through the structural support member 12. Coupling of the first cover member 14 and/or the second cover member 16 may be achieved by creating junctions 18 such as by chemical, mechanical or thermal means. For example, the junctions 18 may be formed by welding, adhering using a biocompatible adhesive, or by forming interlocking or interfacing members on opposing surfaces of the support structure 12, the first cover member 14 and/or the second cover member 16 depending upon the surfaces to be coupled. Additionally, junction 18 may be formed by mechanical interference between the support structure 12 and the first cover member 14 and/or the second cover member 16.

As illustrated with more particularity with reference to FIGS. 3-5, the first cover member 14 and the second cover member 16 may consist of dual discrete members as depicted in FIG. 4. In this version of the first embodiment, each of the first cover member 14 and the second cover member 16 terminate with opposing proximal 24 and distal 26 ends of the respective first cover member 14 and second cover member 16. Junctions 18 may be provided either between the structural support member 12 and the proximal 24 and distal 26 ends of the first 14 and second 16 cover members, between opposing wall surfaces of the first 14 and second 16 cover members and through interstitial spaces in the structural support member 12, or both. Thus, the junctions 18 may occur between the cover members 14, 16 and the structural support member, between the cover members 14, 16 only, or both.

Alternatively, as depicted in FIG. 3, the first cover member 14 and the second cover member 16 may consist of either a single cover member that is positioned adjacent both the first and second wall surfaces of the structural support member and has an eversion region 22 positioned at either a proximal or distal end of the structural support member. Alternatively, dual cover members may be employed with a junction between the first cover member 14 and the second cover member 16 being formed therebetween at the eversion region 22. In accordance with yet another variation of the first embodiment of the invention illustrated in FIG. 5, the first cover member 14 and the second cover member 16 may consist of a single cover member 14 that is everted over both the proximal end 22 of the structural support member 12 and over the distal end 28 of the structural support member 12 and opposing ends 30, 32 of the first cover member 14 are either conjoined to one another, coupled to the structural support member 12 or joined at a junction region 18 to an opposing surface of the first cover member 14.

Each of the first 14 and second 16 cover members preferably has a plurality of openings 15 passing there through. Each of the plurality of openings 15 preferably has a pore size within the range of 0.1 μm to 1000 μm in at least one of an x or y-axis of the opening, with the total open surface area of the first 14 and second 16 cover being between 0.001 to 90% and which permit cellular and sub-cellular physiological matter, such as proteins, to pass through the openings 15 without permitting fluid seepage there through. As used herein, the term “pore size” is intended to connote a dimension in at least one of an x-axis or a y-axis of the opening 15. The total open surface area of the first 14 or second 16 cover may be calculated by dividing the surface area of each the plurality of openings 15 by the total surface area on either the lumenal or ablumenal surface of the first 14 or second 16 cover member. The plurality of openings 15 also impart dimensional flexibility to the first 14 and second 16 cover members and permits flexibility, compressibility and expandability along the longitudinal axis of the stent-graft device 10, while also permitting compliance, foldabilty and expandability in the radial axis of the stent-graft device 10. The plurality of openings 15 are preferably provided in a pattern array in order to maximize the physical properties of the first 14 and second 16 cover members and, hence, the resulting inventive stent-graft 10. For example, the pattern array may be provided to selectively enhance longitudinal flexibility while reinforcing against radial compliance.

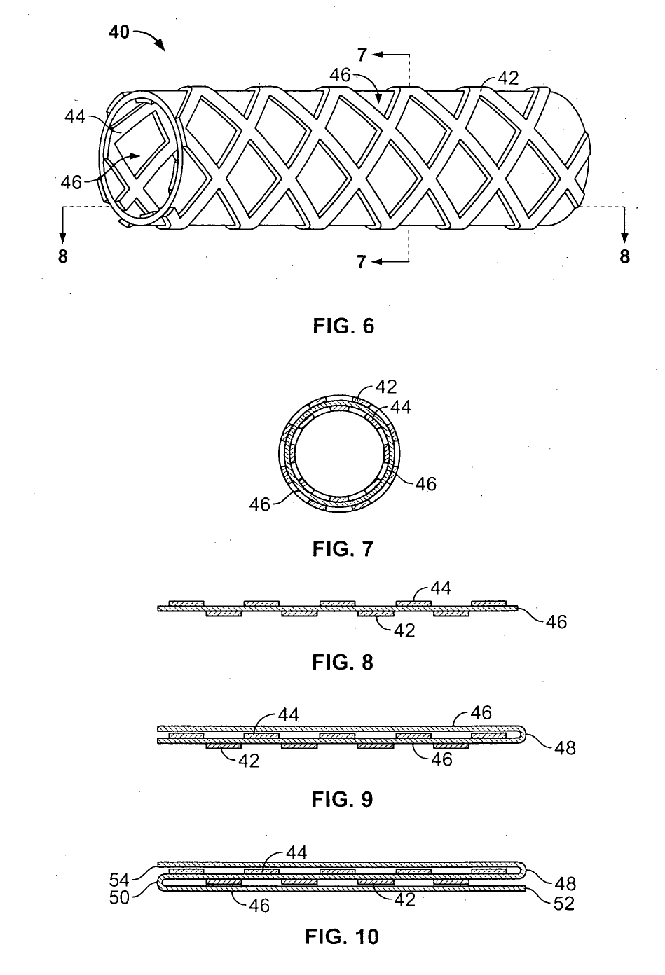

A second embodiment of the inventive stent-graft 40 is illustrated with reference to FIGS. 6-10. Stent-graft 40 consists generally of at least a first structural support member 42 and a second structural support member 44, which are for example tubular stent members, and a metallic cover member 46 having a plurality of openings 45. The at least two structural support 42, 44, for ease of reference, will be referred to as stent members 42, 44. Stent members 42, 44 are preferably generally tubular in configuration, and may be formed as tubular members or initially as planar members that are rolled into a tubular configuration. Alternatively, where it is desirable that the first and second structural support members 42, 44 not constitute stents, the first and second structural support members 42, 44 may be configured to define alternative geometries suitable for a particular application. Such alternative geometries may include, for example, planar geometries for use as patches, frustroconical geometries such as for use as anchors for dental implants or other complex geometries such as for osteal implants.

Where the first structural support member 42 and the second structural support member 44 are selected as stents, the stent members 42, 44 are preferably positioned concentrically relative to one another. The metallic cover member 46 is then positioned concentrically intermediate the first and second stent members 42, 44 along at least a portion of a longitudinal axis of the first and second stent members 42, 44.

The first 42 and second 44 structural support members may either be joined, as described above, to the metallic cover member 46 or may be joined to one another outside the surface area of the metallic cover member 46.

A plurality of openings 45 is provided and passes through the thickness of the cover member 46. As with the first embodiment of the invention, each of the plurality of openings 45 preferably has an pore size within the range of 0.1 μm to 1000 μm, with the total open surface area of the graft being between 0.001 to 90% and which permit cellular and sub-cellular physiological matter, such as proteins, to pass through the openings 45 without permitting fluid seepage there through. Both the pore size of the openings 45 and the total open area of the cover member 46 may be selected in view of the following non-exclusive factors: the desired flexibility of the cover member 46, the desired hoop strength of the cover member 46, the desired degree of geometric enlargement due to deformation of the openings 45 and the desired delivery profile size.

The inventive cover member 46 may be fabricated of pre-existing conventionally produced wrought materials, such as stainless steel or nitinol hypotubes, or may be fabricated by thin film vacuum deposition techniques. In addition to wrought materials that are made of a single metal or metal alloy, the inventive grafts may be comprised of a monolayer of biocompatible material or of a plurality of layers of biocompatible materials formed upon one another into a self-supporting laminate structure. Laminate structures are generally known to increase the mechanical strength of sheet materials, such as wood or paper products. Laminates are used in the field of thin film fabrication also to increase the mechanical properties of the thin film, specifically hardness and toughness. Laminate metal foils have not been used or developed because the standard metal forming technologies, such as rolling and extrusion, for example, do not readily lend themselves to producing laminate structures. Vacuum deposition technologies can be developed to yield laminate metal structures with improved mechanical properties. In addition, laminate structures can be designed to provide special qualities by including layers that have special properties such as superelasticity, shape memory, radio-opacity, corrosion resistance etc.

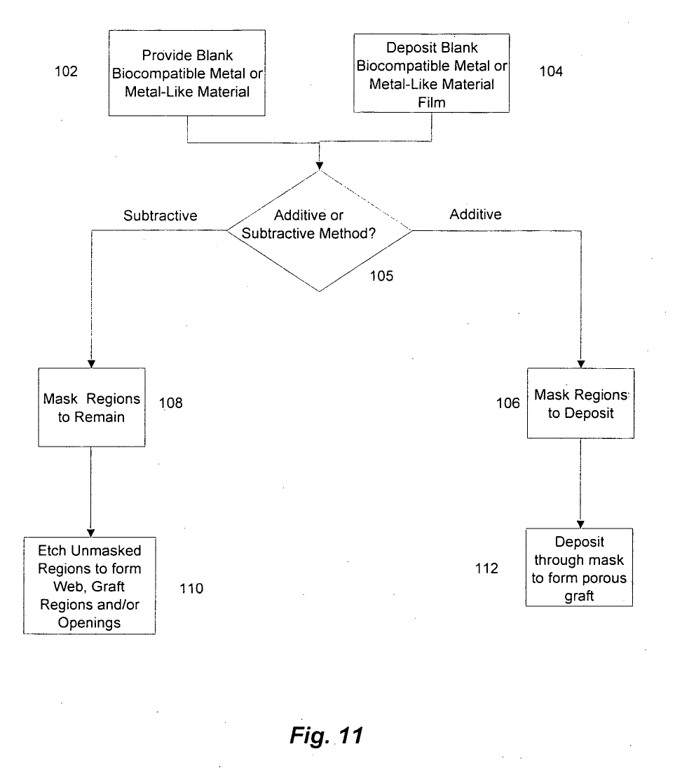

According to the preferred method of making the graft of the present invention, the graft is fabricated of vacuum deposited metallic and/or pseudometallic films. With particular reference to FIG. 11, the fabrication method 100 of the present invention is illustrated. A precursor blank of a conventionally fabricated biocompatible metal or pseudometallic material may be employed at step 102. Alternatively, a precursor blank of a vacuum deposited metal or pseudometallic film may be employed at step 104. A decision 108 is made whether to process the precursor blank from step 102 or step 104 by either subtractive or additive processing is made. If a subtractive process is to be employed, the precursor blank material obtained either from step 102 or step 104 may then be masked at step 108 leaving exposed only those regions which will define a plurality of openings and which will be removed to form the openings. The exposed regions from step 108 are then subjected to removal at step 110, either by etching, such as by wet or dry chemical etching processing, with the etchant being selected based upon the material of the precursor blank, or by machining, such as by laser ablation or EDM. Alternatively, when employing the vacuum deposition step 104, a pattern mask corresponding to the plurality of openings to be formed later and with openings to permit deposition of the graft material through the mask, may be interposed at step 106 between the target and the source and the metal or pseudometal deposited at step 112 through the pattern mask to form the graft material with openings corresponding to the masked regions. Further, when employing the vacuum deposition step 104, plural film layers maybe deposited to form a laminate film structure of the film prior to or concurrently with forming the plurality of openings.

Where a laminate film is fabricated as the graft, it is necessary to provide for good adhesion between the layers. This may be achieved by providing for a relatively broad interfacial region rather than for an abrupt interface. The width of the interface region may be defined as the range within which extensive thermodynamic parameters change. This range can depend on the interface area considered and it may mean the extent of interface microroughness. In other words, adhesion may be promoted by increased interfacial microroughness between adjacent layers within the film. The microroughness may be imparted by chemical or mechanical means, such as chemical etching or laser ablation, or may be included as a process step during vacuum deposition by selectively depositing a metal or pseudometallic species to form the microroughness.

Thus, the present invention provides a new metallic and/or pseudometallic implantable graft that is biocompatible, geometrically changeable either by folding and unfolding or by application of a plastically deforming force, and capable of endoluminal delivery with a suitably small delivery profile. Suitable metal materials to fabricate the inventive covers are chosen for their biocompatibility, mechanical properties, i.e., tensile strength, yield strength, and their ease of deposition include, without limitation, the following: titanium, vanadium, aluminum, nickel, tantalum, zirconium, chromium, silver, gold, silicon, magnesium, niobium, scandium, platinum, cobalt, palladium, manganese, molybdenum and alloys thereof, such as chromium-cobalt alloy, zirconium-titanium-tantalum alloys, nickel-titanium, and stainless steel. Examples of pseudometallic materials potentially useful with the present invention include, for example, composite materials, ceramics, quartz, and borosilicate.

The present invention also provides a method of making the inventive stent-graft devices by vacuum deposition of a graft-forming metal or pseudometal and formation of the openings either by removing sections of deposited material, such as by etching, EDM, ablation, or other similar methods, or by interposing a pattern mask, corresponding to the openings, between the target and the source during deposition processing. Alternatively, a pre-existing metal and/or pseudometallic film manufactured by conventional non-vacuum deposition methodologies, such as wrought hypotube, may be obtained, and the openings formed in the pre-existing metal and/or pseudometallic film by removing sections of the film, such as by etching, EDM, ablation, or other similar methods. An advantage of employing laminated film structures to form the inventive graft is that differential functionalities may be imparted in the discrete layers. For example, a radiopaque material such as tantalum may form one layer of a structure while other layers are chosen to provide the graft with its desired mechanical and structural properties.

While the present invention has been described with reference to its preferred embodiments, those of ordinary skill in the art will understand and appreciate that variations in materials, dimensions, geometries, and fabrication methods may be or become known in the art, yet still remain within the scope of the present invention which is limited only by the claims appended hereto.

What is claimed is:

1. A medical device, comprising:
(a) A generally tubular structural support member having luminal and abluminal wall surfaces thereof and being comprised of at least one of a metallic and pseudometallic material; and
(b) At least one of a plurality of generally tubular cover members comprised of at least one of a metallic and pseudometallic material, the at least one of a plurality of generally tubular cover members being concentrically adjacent both the luminal and abluminal wall surfaces of the generally tubular structural support member.

2. The medical device according to claim 1, wherein the at least one metallic and pseudometallic material further comprises a thin metallic film.

3. The medical device according to 2, wherein the thin metallic film is selected from the group consisting of titanium, vanadium, aluminum, nickel, tantalum, zirconium, chromium, silver, gold, silicon, magnesium, niobium, scandium, platinum, cobalt, palladium, manganese, molybdenum and alloys thereof, chromium-cobalt alloy, zirconium-titanium-tantalum alloy, nickel-titanium alloy, and stainless steel.

4. The medical device according to claim 1, wherein the at least one metallic and pseudometallic material further comprises a pseudometallic material selected from the group consisting of ceramic, quartz, borosilicate, carbon fibers, boron fibers, boron carbide fibers, carbon nanotubes, carbon and graphite fibers, silicon carbide fibers, steel fibers, tungsten fibers, graphite/copper fibers, titanium and silicon carbide/titanium fibers.

5. The medical device according to claim 1, wherein the at least one of a plurality of generally tubular cover members further comprises a plurality of openings passing through the tubular cover members.

6. The medical device according to claim 5, wherein each of the plurality of openings has a pore size between about 0.1 μm to 1000 μm in either an x-axis or a y-axis of the opening.

7. The medical device according to claim 5, wherein the plurality of microporous openings form a total open surface area in the generally tubular graft member between about 0.001 to 90% of one of an ablumenal or luminal surface area of the generally tubular cover member.

8. The medical device according to claim 1, wherein the at least one of a plurality of generally tubular cover members are coupled to at least one of a proximal and distal end of the structural support member.

9. The medical device according to claim 1, further comprising a luminal cover member and an abluminal cover member attached to each other through interstices in the generally tubular structural support member.

10. The medical device according to claim 1, further comprising at least one affixation between at least one of the at least one generally tubular cover member and the generally tubular structural support member or the at least one generally tubular cover member.

11. The medical device according to claim 10, wherein the at least one affixation is selected from the group consisting of: chemically, mechanically or thermally formed affixation.

12. The medical device according to claim 11, wherein the mechanically formed affixation further comprises an interference fit.

13. The medical device according to claim 11, wherein the mechanically formed affixation further comprises an interfacing detent and trough.

14. The medical device according to claim 8, wherein the at least one of a plurality of generally tubular graft members are coupled to the at least one of a proximal and distal end of the structural support member by a junction created by at least one of chemical, thermal and mechanical means.

15. The medical device according to claim 9, wherein the luminal and abluminal graft members one attached to one another by at least one of chemical, thermal and mechanical means.

16. The medical device according to claim 9, wherein the interstices are capable of geometric deformation about the attachment between the luminal and the abluminal graft members.

17. The medical device according to claim 1, further comprising a defined junction region on each of the generally tubular structural support member and the at least one generally tubular cover member in alignment with one another and a junction formed therebetween.

18. A covered stent device, comprising:
(a) A diametrically expandable stent; and
(b) At least one cover member consisting essentially of a thin metallic film having a plurality of openings passing therethrough, and concentrically adjacent at least one of a luminal and an abluminal wall surface of the diametrically expandable stent.

19. The covered stent device, according to claim 18, wherein the thin metallic film is selected from the group consisting of titanium, vanadium, aluminum, nickel, tantalum, zirconium, chromium, silver, gold, silicon, magnesium, niobium, scandium, platinum, cobalt, palladium, manganese, molybdenum and alloys thereof, such as chromium-cobalt alloy, zirconium-titanium-tantalum alloys, nickel-titanium alloy, and stainless steel.

20. The covered stent device according to claim 18, wherein the at least one cover member is concentrically carried adjacent each of the luminal and abluminal wall surfaces of the diametrically expandable stent, wherein the at least one cover member adjacent the luminal wall surface is coupled to the at least one cover member adjacent the abluminal wall surface through interstitial openings in the diametrically expandable stent in such a manner as to permit geometric deformation of the interstitial openings.

21. The covered stent device according to claim 18, wherein the at least one cover member is coupled to the at least one of a proximal and distal end of the diametrically expandable stent.

22. The covered stent device according to claim 18, wherein the coupling is created by at least one of chemical, thermal and mechanical means.

23. The covered stent device according to claim 21, wherein the coupling is created by at least one of chemical, thermal and mechanical means.

24. The covered stent according to claim 22, wherein each of the plurality of openings has a pore size between about 0.01 μm to about 1000 μm.

25. The covered stent according to claim 18, wherein the plurality of openings form a total open surface area in the generally tubular graft member between about 0.001 to 90% of one of an ablumenal or luminal surface area of the generally tubular cover member.

26. The covered stent device according to claim 18, further comprising a polymeric material capable of releasing a pharmacologically active agent therefrom disposed on at least one of the diametrically expandable stent or the at least one cover member, whereby the polymeric material does not subtend the openings in the at least one cover member or interstices in the diametrically expandable stent.

27. A medical device, comprising:
(a) A generally tubular member having luminal and abluminal wall surfaces thereof and being comprised of at least one of a metallic and pseudometallic material; and
(b) At least one of two structural support members, a first structural support member being positioned concentrically adjacent a luminal wall surface of the generally tubular graft member and a second structural support member being positioned concentrically adjacent an abluminal wall surface of the generally tubular graft member.

28. The medical device according to claim 27, wherein the at least one metallic and pseudometallic material further comprises a thin metallic film.

29. The medical device according to 27, wherein the thin metallic film is selected from the group consisting of titanium, vanadium, aluminum, nickel, tantalum, zirconium, chromium, silver, gold, silicon, magnesium, niobium, scandium, platinum, cobalt, palladium, manganese, molybdenum and alloys thereof, chromium-cobalt alloy, zirconium-titanium-tantalum alloy, nickel-titanium alloy, and stainless steel.

30. The medical device according to claim 27, wherein the at least one metallic and pseudometallic material further comprises a pseudometallic material selected from the group consisting of ceramic, quartz, borosilicate, carbon fibers, boron fibers, boron carbide fibers, carbon and graphite fibers, carbon nanotubes, silicon carbide fibers, steel fibers, tungsten fibers, graphite/copper fibers, titanium and silicon carbide/titanium fibers.

31. The medical device according to claim 27, wherein the generally tubular members further comprises a plurality of openings passing through the tubular graft member.

32. The medical device according to claim 27, wherein the at least two structural support members are each coupled to the generally tubular member at least at one of a proximal and distal end of the generally tubular member.

33. The medical device according to claim 27, wherein the at least two structural support members are coupled to one another.

34. The medical device according to claim 27, wherein each of the plurality of openings has dimension between about 0.1 μm to 1000 μm in either of an x-axis or y-axis of the opening.

35. The medical device according to claim 27, wherein the plurality of openings form a total open surface area in the generally tubular member between about 0.001 to 90% of the surface area of the generally tubular member.
